(19) United States
(12) Patent Application Publication
(10) Pub. No.: US 2004/0042984 A1
(43) Pub. Date: Mar. 4, 2004

(54) Skin whitening composition containing arbutin and glucosidase as active ingredients

(76) Inventors: Deok Hoon Park, Yongin-si (KR); Dong II Jang, Anyang-si (KR); Kuk Hyun Kim, Seongnam-si (KR)

Correspondence Address: STAAS & HALSEY LLP, SUITE 700, 1201 NEW YORK AVENUE, N.W., WASHINGTON, DC, 20005, US

(21) Appl. No.: 10/416,311

(22) PCT Filed: Dec. 28, 2001
(86) PCT No.: PCT/KR01/02285

(30) Foreign Application Priority Data
Jan. 10, 2001 (KR) 2001/1236

(51) Int. Cl. A61K 7/00; A61K 35/78; A61K 38/47

(52) U.S. Cl. 424/62; 424/94.61; 424/725

(57) ABSTRACT

The present invention relates to skin whitening and/or depigmenting composition containing arbutin and glucosidase as active ingredients. The glucosidase is an enzyme hydrolyzing arbutin into hydroquinone and glucose.

In the composition of this invention, arbutin and glucosidase are separated and mixed just before applying to the skin. Then arbutin hydrolyzes into hydroquinone and glucose and the whitening effects are achieved by the hydroquinone inhibiting melanogenesis. The composition of this invention showed the superiority in safety and stability.

### TECHNICAL FIELD

The present invention relates to skin whitening compositions comprising arbutin and glucosidase. More particularly, the arbutin depicted in the chemical formula 1 and glucosidase hydrolyzing arbutin into hydroquinone and glucose are contained in a formula. Otherwise, arbutin and glucosidase are separated and mixed just before applying to the skin.

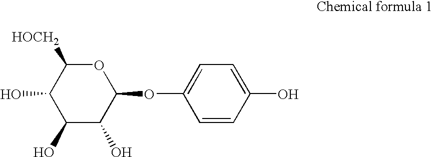

### BACKGROUND ART

Generally, skin pigmentation is generated due to the increased melanin synthesis in UV irradiated melanocyte in the skin. Melanin is synthesized by the consecutive oxidation of tyrosine. Tyrosine is catalyzed by glucosidase, the oxidative enzyme, and transformed into DOPAquinone. The DOPAquinone spontaneously proceed to melanin. Melanin absorbs solar ultraviolet rays for protection but it produces skin color disorder such as melasma, dark spots at specific regions. For the purpose of skin whitening, the depigmenting compositions are contained in the cosmetics.

Primarily, depigmentation in the skin can be achieved by inhibiting some step of melanogenesis process. Hydroquinone, arbutin, kojic acid, ascorbic acid and the derivatives thereof are adapted to depigment in the skin. Plant extracts from bearberry (Korean Patent Publication No. 2000-035398), mulberry and apple (Korean Patent No. 1998-147412) containing the above ingredients are contained in cosmetics and for skin depigmentation.

However, depigmenting agents such as ascorbic acid, kojic acid, glutathione, mulberry extracts are apt to lose the stabilities and induce skin irritation. Furthermore, kojic acid and its derivatives, one of the most potent tyrosinase inhibitors, cost high to produce depigmenting agents in a large scale. The solar light and heat may induce decoloration of arbutin and its efficacy inhibiting tyrosinase decreases. Therefore, the arbutin is recognized to have less effect on depigmentation than hydroquinone.

Hydroquinone, the most powerful whitening compound, is widely used at nonprescription ointment or prescriptions of dermatologist. Many clinical researches of hydroquinone which has depigmenting effect against hyperpigmentation such as melasma (Arndt et al., JAMA. 194: 965-967, 1965) have been reported. Hydroquinone, depicted in the chemical formula 2, inhibits tyrosinase, an essential enzyme involved in the melanogenesis process. In particular, tyrosinase catalyzes the conversion of tyrosine into dihydroxyphenylalanine (dopa) by its hydroxylase activity, and the conversion of dopa into dopaquinone by virtue of its oxidase activity.

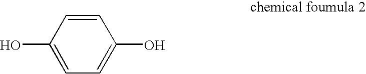

Although hydroquinone has a certain efficacy, it is unstable and reactive compound and easily leads to decoloration in cosmetic formula such as micelle, emulsion. Unfortunately even low concentration of hydroquinone could induce allergic contact dermatitis and acute or chronic irritation on skin (Choudat et al., J. Ind. Med. 45(6): 376-80, 1988). To avoid such side effects, the dosage should be stringently controlled according to the susceptibility of patients (Pearl E. et al., Archi. Dermatol. 131:1453-1457, 1995)

Arbutin is a glycoside of hydroquinone which is linked with beta-D-glucose. Although arbutin is being used for whitening cosmetics, it is not easily absorbed into the skin and has less whitening effect than hydroquinone. The effect of arbutin inhibiting tyrosinase activity is about 1/1,000 of hydroquinone. (Kazuhisa Maeda and Minoru Fukuda, Arbutin: Mechanism of its depigmenting action in human melanocyte culture. J. Pharm. Exp. Therap. 1996; 276(2): 765-769).

A method using surfactant adding nitrogen has been used to enhance skin absorption of arbutin (U.S. Pat. No. 5,759,528). Many researchers and studies for development of new whitening derivates have been proceeded but little successful reports has been released yet. In conclusion, arbutin has not been used widely since arbutin has lower skin absorption rate, lower whitening effect than hydroquinone although arbutin is more stable and has less skin trouble.

Presently, hydroquinone is the most effective whitening composition. Due to its side effects such as skin toxicity and instability a novel effective whitening composition with low skin irritation and high stability is required.

In order to overcome the foregoing and other disadvantages, the present invention provides a novel skin whitening composition minimizing skin irritation and maximizing stability by mixing arbutin and glucosidase just before applying to the skin.

### DISCLOSURE OF INVENTION

The object of the present invention is to provide a non-toxic, non-irritating and non-allergenic formula that shows high effect on depigmentation in human skin.

Another object of the present invention is to provide skin whitening composition containing arbutin depicted in chemical formula 1 and glucosidase as active compounds.

The arbutin content of the composition can be adjusted to 0.05˜5.0% in the total amount preferably, 0.1˜3.5%. The glucosidase content of the composition can be adjusted to 75˜150 units on the basis of 3% of arbutin. Insufficient concentration of arbutin or glucosidase may cause not enough whitening effect and too much arbutin and glucosidase may cause skin irritation.

The above-mentioned arbutin can be natural arbutin, chemical arbutin or the arbutin that is biotechnologically made. Preferably, Natural arbutin isolated from bearberry leaf or bearberry extracts itself can be used in this invention.

Glucosidase is an enzyme hydrolyzing glucoside linkage into glucose and aglycon. Glucosidase is widely found in yeasts, bacteria, fungi and the digestive organs of animals. Recently the gene of glucosidase is cloned and the glucosidase can be produced biotechnologically. The sources of glucosidase for embodying this invention described above are not restricted. According to this invention, the plant or microorganism extracts including glucosidase is added to the cosmetics. Preferably the said plant can be almond, barley or oats and the said microorganism can be Aspergillus niger. The origins of glucosidase hydrolyzing arbutin into hydroquinone described in this invention are not limited.

In the composition of this invention, arbutin and glucosidase are separated and mixed just before applying to the skin. Then arbutin hydrolyzes into hydroquinone and glucose and the whitening effects are achieved by the hydroquinone inhibiting melanogenesis. The composition of this invention showed the superiority in safety and stability.

The compositions of the present invention may be added to the cosmetics such as a toner, a lotion, a gel, an emulsion, a cream, and an unguent. The preservatives, the anti-oxidants, the coloring matters, the perfumes, the tensio-active agents, the thickening agents, humectants, UV absorber or surfactants can be added to the above form of composition at need. The volume of activity adjuvants and/or another active ingredients is preferably adjusted to 0.01˜20% in the total amount.

The composition of the present invention are intended for applying to the localized pigmented lesion, which generally can be provided for the medical purposes in the form of solution, gel, simple or complex emulsion, microcapsule or liposome. In case that a composition comprises arbutin and glucosidase in a formula, one of the ingredients, preferably glucosidase can be entrapped into microcapsule, liposome or lipid vesicle to hinder the hydrolysis reaction until they applied to the skin. Therefore, the stability could be maximized.

The thin Layer Chromatography (TLC) method that is generally used in organic material analysis was utilized to confirm complete hydrolysis of arbutin into hydroquinone and glucose by glucosidase.

To confirm that the composition comprising arbutin and glucosidase is more stable than hydroquinone only composition, the reaction mixture comprising arbutin and glucosidase was incubated at 37° C. for 10 minutes and the reaction was terminated by cooling down in the ice water. The absorbency was measured at 400 nm and the degree of discoloring can be expressed in the ratio of the extent of arbutin solution to that of hydroquinone solution. It was confirmed that the stability of the mixture composition comprising arbutin and glucosidase is superior to that of hydroquinon. as the glucosidase concentration decrease, the stability increase since the hydrolysis is accelerated in proportion to the glucosidase concentration.

In another preferred embodiments of this invention, the safety of this composition was tested by skin-irritation against the occlusive patch applied to healthy-adult volunteers. As a result, it is approved that the mixture composition comprising arbutin and glucosidase did not show any irritation in spite of releasing hydroquinone. However, the same concentration of hydroquinone composition as a controlled experiment showed the positive irritation. It is interpreted that the skin is more adaptable to the gradually increasing amount of irritant than the high concentration of irritant at a time.

Further, the composition comprising arbutin and glucosidase is applied to the pigmented lesion that is artificially induced by UV irradiation on the skin of arm of volunteers. The result is the composition comprising arbutin and glucosidase showed more effective than the composition comprising arbutin only or comprising hydroquinone only.

### BRIEF DESCRIPTION OF THE DRAWINGS

FIG. 1 shows Thin Layer Chromatography (TLC) to confirm that arbutin is hydrolyzed to hydroquinone and glucose by glucosidase.

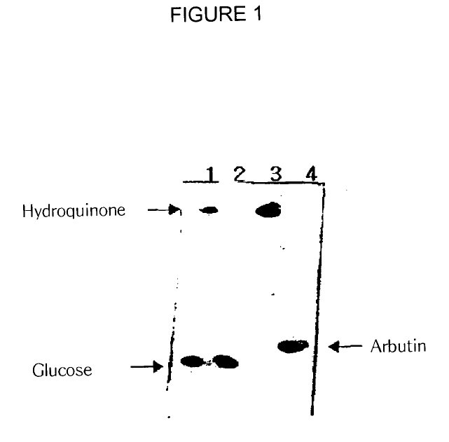

1. arbutin+glucosidase

2. glucose

3. hydroquinone

4. arbutin

### BEST MODE FOR CARRYING OUT THE INVENTION

Practical and presently preferred embodiments of the present invention are illustrative as shown in the followings.

However, it will be appreciated that those skilled in the art, on consideration of this disclosure, may make modifications and improvements within the scope of the present invention.

Preferred Embodiments 1: Hydrolyzed Products from Arbutin by Glucosidase

0.05% arbutin (sigma, USA) and 20unit/ml beta-1,4-glucosidase (sigma, USA, EC 3.2.1.21) were reacted at 37° C. for 30 minutes in 0.1M sodium acetate buffer (pH 5.0). After the reaction, hydroquinone production was determined using Thin Layer Chromatography in the running solvent which is a mixture of n-buthanol, ethanol, water of 5:3:2.

Then, the TLC plate is dried and sprayed with charring solution (10% CuSO4 in 8% H3PO4) and immediately dried with heat-gun. The Rf values of spots are calculated.

According to the Table 1, only hydroquinone and glucose were observed in the TLC. Therefore, complete hydrolysis of arbutin by glucosidase is inferred.

TABLE 1

| Materials | Rf value |
|---|---|
| Arbutin | 0.2 |
| Hydroquinone | 0.9 |
| Glucose | 0.1 |
| Arbutin + Glucosidase | 0.9, 0.1 |

*Rf = running distance of solute/whole distance spread by the solvent

Preferred Embodiment 2: Examination of Stability of the Mixed Composition

The formula stability of arbutin-glucosidase mixed formula compared with the hydroquinone only formula was examined by the decoloration. 100 mM arbutin and each 1, 10 and 20 unit/ml glucosidase were reacted in 1 ml of sodium acetate buffer (pH 5.0) at 37° C. for 10 minutes. The reaction was terminated by cooling down in the ice water. Then its mixed 1 ml of cold 20% Trichloroacetic acid (TCA) was added to remove glucosidase activity. The mixture was centrifuged at 12000 rpm. The supernatant solution incubated at 37° C. for 24 hours and the absorbency was measured at 400 mm. The degree of decoloration of hydroquinone was regarded as 100% and the ratio of other samples compared to hydroquinone were calculated. As the control, 100 mM arbutin and 100 mM hydroquinone solution (sigma, USA) was used. In the Table 2, as the glucosidase concentration decrease, the stability increase since the hydrolysis is accelerated in proportion to the glucosidase concentration.

TABLE 2

| | | Discoloration rate |
|---|---|---|
| | Absorbance | (% of decoloring of |
| Materials | (400 nm) | hydroquinone solution) |
| Buffer solution | 0.002 | 0.5% |
| Hydroquinone | 0.356 | 100% |
| solution | | |
| Arbutin solution | 0.005 | 1.40% |
| Arbutin + 1 ul | | |
| Glucosidase | 0.012 | 3.37% |
| Arbutin + 10 ul | 0.094 | 26.4% |
| Glucosidase | | |
| Arbutin + 20 ul | | |
| Glucosidase | 0.168 | 47.2% |

*discoloration rate of hydroquinone is regarded as 100% and others are compared with hydroquinone solution

Preferred Embodiment 3: Inhibition Effect of Primary Irritation on the Skin Caused by Arbutin-Glucosidase Reaction

The occlusive patch test was performed to examine skin irritation of the mixture of arbutin and glucosidase by 30 healthy adults. The test patches containing the solution of arbutin and glucosidase of preferred embodiment 2 were applied to the lower part of the volunteers' arms. After 24 hours, the patches were removed. The status of skin was observed from 30 minutes to 48 hours after removing the patches, and the degree of irritation has been classified as described in Table 3a.

Table 3b shows the result of irritation test. While hydroquinone solution elicits very strong skin irritation, arbutin-glucosidase mixed solution hardly shows skin irritation.

TABLE 3a

| Sign | Indication |
|---|---|
| ?+ | doubtful response, an immaterial erythema |
| + | weak response (without vesicle), erythema, papule |
| ++ | strong response (with vesicle), erythema, papule, |
| | vesicle |
| +++ | very strong positive response, bulla |
| −−− | negative |

TABLE 3b

Response

| Material | 24 h | 48 h |
|---|---|---|
| Buffer solution | − | − |
| Hydroquinone solution | +++ | +++ |
| Arbutin solution | − | − |
| Arbutin + 1 ul Glucosidase | − | − |
| Arbutin + 10 ul Glucosidase | − | − |
| Arbutin + 20 ul Glucosidase | − | − |

Preferred Embodiment 4: Skin Whitening Effect Both Arbutin-Glucosidase Mixed Formula

Practical formula based on the preferred embodiments described above was prepared and applied to skin whitening. The formula 1 containing arbutin and formula 2 containing glucosidase were prepared separately, mixed just before application and applied to the skin in the ratio of mixture formula 1 and formula 2, 10:1. Then, each effect was compared.

A panel of 10 adult volunteers aged 25-30 were selected and were enrolled in a 6 week in vivo study. 4 circled pigmented region which is 1.5 cm in diameter were induced on the lower forearm of volunteers by the irradiation of UV light (UV lamp; Philips TL20w/12UV, TM02/09UV) by 1.5 MED each. The pigmentations were performed twice a day for 2 days. The mixture of Formula 1 and Formula 2 in the ratio 10 to 1, as an experimental formula, and the comparing formula 1 to 3 were applied twice a day for 6 weeks. Then, the skin whitening effects were observed. As a result, the formula without depigmenting agents or the formula containing only arbutin shows no skin whitening effects. Although the formula containing hydroquinone has skin whitening effects, it is confirmed to cause skin trouble. On the contrary, the mixture formula comprising arbutin and glucosidase shows better depigmenting effects than any other formula without any side effects.

TABLE 4a

| | | | Compar- | Compar- | Compar- |
|---|---|---|---|---|---|
| | Formu- | Formu- | ing | ing | ing |
| Ingredients (%) | la 1 | la 2 | Formula 1 | Formula 2 | Formula 3 |
| Arbutin | 3.0 | — | — | 3.0 | — |
| Glucosidase | — | 1000 | — | — | — |
| | | unit | | | |
| Hydroquinone | — | — | — | — | 2.0 |
| Glycerine | 10.0 | 1.0 | 10.0 | 10.0 | 10.0 |
| Propylenglycol | 5.0 | 0.5 | 5.0 | 5.0 | 5.0 |
| Cellulose gum | 0.3 | 0.03 | 0.3 | 0.3 | 0.3 |
| Hyaruronic acid | 10.0 | 1.0 | 10.0 | 10.0 | 10.0 |
| pH regulator | fit | fit | fit | fit | fit |
| Deionized water | to 100 | to 100 | to 100 | to 100 | to 100 |

TABLE 4b

| | skin depigmentation effects | | | |
|---|---|---|---|---|
| | | Slightly | Not | Side |
| Formula | Effective | effective | effective | effects |
| Mixture of Formula 1 | 6 | 3 | 1 | — |
| and Formula 2(10:1) | | | | |
| Comparing formula 1 | — | 2 | 8 | — |
| Comparing formula 2 | — | 3 | 7 | — |
| Comparing formula 3 | 5 | 2 | — | 3 |

The following is a preferable example of an essence containing composition according to the present invention.

Mixing Formula 1 with Formula 2 in the ration of 10:1, on the hand, before applying the mixture to the face

### FORMULATION EXAMPLE 1

Essence Preparation Utilizing the Ingredients of this Invention

| | |
|---|---|
| Formula 1 | Formula 2 |

| | Percentage | | Percentage |
|---|---|---|---|
| Ingredient | (%) | Ingredient | (%) |
| Arbutin | 3.0 | Glucosidase | 1000 units |
| Glycerine | 10.0 | Glycerine | 1.0 |
| Propylenglycol | 5.0 | Propylenglycol | 0.5 |
| Cellulose gum | 0.3 | Cellulose gum | 0.03 |
| Hyaluronic acid | 10.0 | Hyaluronic acid | 1.0 |
| pH regulator | fit | pH regulator | fit |
| Fragrance | dash | Fragrance | dash |
| Preservative | dash | Preservative | dash |
| Pigment | dash | Pigment | dash |
| Deionized water | to 100 | Deionized water | to 10 |

### FORMULATION EXAMPLE 2

Oil-in-water Cosmetic Emulsion Preparation Utilizing the Ingredients of this Invention

| Formula 1 | | Formula 2 | |
|---|---|---|---|
| | Percentage | | Percentage |
| Ingredient | (%) | Ingredient | (%) |
| Arbutin | 3.0 | Glucosidase | 1000 units |
| Wax | 3.0 | Wax | 0.3 |
| Liquid paraffin | 4.0 | Liquid paraffin | 0.4 |
| Glycerine | 10.0 | Glycerine | 1.0 |
| Carboxylvinylpolymer | 0.1 | Carboxylvinylpolymer | 0.01 |
| Polysolvate 60 | 1.1 | Polysolvate 60 | 0.11 |
| Propylenglycol | 5.0 | Propylenglycol | 0.5 |
| pH regulator | fit | pH regulator | fit |
| Frgrance, | dash | Frgrance, | dash |
| preservative, pigment | | preservative, pigment | |
| Deionized water | To 100 | Deionized water | to 10 |

### FORMULATION EXAMPLE 3

Pack Preparation Utilizing the Ingredients of this Invention

| Formula 1 | | Formula 2 | |
|---|---|---|---|
| Ingredient | Percent (%) | Ingredient | Percent (%) |
| Arbutin | 3.0 | Glucosidase | 1000 units |
| Polyvinylalcohol | 14.0 | Polyvinylalcohol | 1.4 |
| Glycerine | 10.0 | Glycerine | 1.0 |
| Cellulose gum | 0.3 | Cellulose Gum | 0.03 |
| PEG 4000 | 1.0 | PEG 4000 | 0.1 |
| Propylenglycol | 5.0 | Propyleneglycol | 0.5 |
| pH regulator | fit | pH regulator | fit |
| Frgrance, | dash | Frgrance, | dash |
| preservative, | | preservative, | |
| pigment | | pigment | |
| Deionized water | to 100 | Deionized water | to 10 |

### FORMULATION EXAMPLE 4

Nutrient Cream Preparation Utilizing the Ingredients of this Invention

| Formula 1 | | Formula 2 | |
|---|---|---|---|
| Ingredient | Percent (%) | Ingredients | Percent (%) |
| Arbutin | 3.0 | Glucosidase | 1000 untis |
| Liquid paraffin | 10.0 | Liquid paraffin | 1.0 |
| Propylenglycol | 5.0 | Propylenglycol | 0.5 |
| wax | 7.0 | Beeswax | 0.7 |
| Glycerine | 10.0 | Glycerine | 1.0 |
| Polysorbate 60 | 1.3 | Polysorbate 60 | 0.13 |
| pH regulator | fit | pH regulator | fit |
| Frgrance, | dash | Frgrance, | dash |
| preservative, | | preservative, | |
| pigment | | pigment | |
| Deionized water | to 100 | Deionized water | to 10 |

Industrial Applicability

The skin whitening agents of this invention comprises arbutin and glucosidase, which gradually generate hydroquinone by the hydrolysis reaction of glucosidase. The depigmenting effects of the present invention are higher than that of hydroquinone without any skin trouble and unnecessary reaction during storage. Therefore, this invention can be applied to the whitening products with a cosmetic or dermatological or pharmaceutical composition.

What is claimed is:

1. A composition for depigmenting skin, comprising both arbutin and glucosidase as active ingredients

2. A composition for skin depigmenting according to claim 1, which comprises 0.05˜5.0% of arbutin and 75˜150 units of glucosidase per 3% arbutin.

3. A composition for skin depigmenting according to claim 1, wherein said arbutin is selected from the natural arbutin extracted from plant or chemical arbutin synthesized by chemical synthesis.

4. A composition for skin depigmenting according to claim 3, wherein the natural arbutin includes bearberry leaf extracts.

5. A composition for skin depigmenting according to claim 1, wherein glucosidase is isolated from plant or microorganism extracts containing the said glucosidase.

6. A composition for skin depigmenting according to claim 5, wherein said plant is selected from the group consisting of almond, barley and oat.

7. A composition for skin depigmenting according to claim 6, wherein said microorganism is Aspergillus niger.

8. A composition for skin depigmenting according to claim 1, wherein arbutin and glucosidase are physically separated in a container and mixed to initiate the enzyme reaction just before applying to the skin.

9. A composition for skin depigmenting according to claim 1, wherein glucosidase is contained in capsules that are available to the cosmetics and arbutin is including the capsuled glucosidase in a formula.

10. A composition for skin depigmenting according to claim 1, which is applied to the cosmetics.

11. A composition for skin depigmenting according to claim 1, which is applied to the external ointment.

12. A composition for skin depigmenting according to claim 10, wherein the said composition is applied to the forms nutrient cream, toner, lotion, massage cream, eye cream, eye essence, cleansing form, cleansing cream, powder, body lotion, body cream, body oil, body essence, pack.
